(19) United States
(12) Patent Application Publication
(10) Pub. No.: US 2007/0224158 A1
(43) Pub. Date: Sep. 27, 2007

(54) Method for the Cosmetic Treatment of Wrinkled Skin Using a Cosmetic Composition Containing a Tightening Agent and a Dispersion of Solid Particles of a Grafted Acrylic polymer

(75) Inventors: Guillaume Cassin, Villebon / Yvette (FR); Marco Vicic, Bry Sur Marne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/591,583

(22) PCT Filed: Mar. 18, 2005
(86) PCT No.: PCT/FR05/50174

§ 371 Date: Sep. 5, 2006

(30) Foreign Application Priority Data
Mar. 19, 2004 (FR) 0450556

(51) Int. Cl. A61K 31/74

(52) U.S. Cl. 424/78.03

(57) ABSTRACT

The present invention relates to a cosmetic process for softening the wrinkles of wrinkled skin comprising a stage consisting in applying, to said wrinkled skin, a cosmetic composition, in particular an anti-wrinkle composition, comprising, in a physiologically acceptable medium suitable for topical application to the skin of the face: from 0.1 to 20% by weight of at least one tensioning agent, with respect to the total weight of the composition, and at least one dispersion in a liquid fatty phase of solid particles of a grafted ethylenic polymer. This composition makes it possible to contribute a persistent tensioning effect to the keratinous substances to which it is applied.

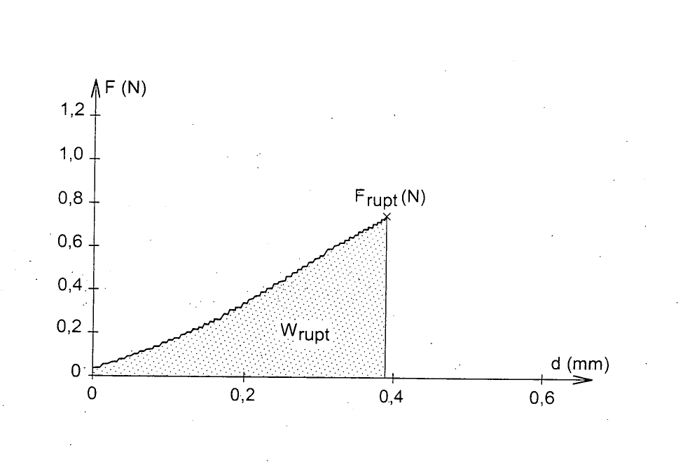

### TECHNICAL FIELD

The present invention relates to a cosmetic process for softening the wrinkles of wrinkled skin employing a cosmetic composition, in particular an anti-wrinkle composition, comprising, in a physiologically acceptable medium suitable for topical application to the skin of the face, from 0.1% to 20% by weight of a tensioning agent, with respect to the total weight of the composition, and a dispersion of a specific ethylenic polymer capable of rendering persistent the tensioning effect brought about by the abovementioned tensioning agent.

The present invention relates to the use of a dispersion of a specific ethylenic polymer for improving the persistence of the tensioning effect provided by a tensioning agent.

The present invention also relates to the use of a dispersion of a specific ethylenic polymer in a composition comprising, as tensioning agent, a colloidal dispersion of inorganic particles for preventing whitening of the skin.

The general field of the invention is thus that of the ageing of the skin.

During ageing of the skin, various signs appear, being reflected in particular by a modification in the skin structure and skin functions. One of these main signs is the appearance of deep wrinkles and fine lines, the scale and the number of which increase with age. The microrelief of the skin becomes less uniform and exhibits an anisotropic nature.

### PRIOR STATE OF THE ART

It is normal to treat these signs of ageing with cosmetic compositions comprising active principles capable of combating ageing, such as α-hydroxy acids, β-hydroxy acids and retinoids. These active principles act in particular on wrinkles by removing the dead cells from the skin and by accelerating the process of cell replacement. However, these active principles exhibit the disadvantage of being effective in the treatment of wrinkles only after a certain application time, namely a time which can range from a few days to several weeks.

In point of fact, current requirements are increasingly tending towards the production of compositions which make it possible to obtain an immediate effect, resulting rapidly in smoothing out of wrinkles and/or fine lines and in the disappearance, even temporary, of signs of fatigue. Such compositions are compositions comprising tensioning agents. It is specified that the term “tensioning agent” is understood to mean compounds capable of having a tensioning effect, that is to say which can tighten the skin and bring about an immediate reduction in, indeed even disappearance of, its wrinkles and fine lines.

These tensioning agents can in particular be polymers of natural or synthetic origin in aqueous dispersion capable of forming a film bringing about the retraction of the stratum corneum, that is to say the surface horny layer of the epidermis. The cosmetic or dermatological use of such polymer systems for softening the effects of ageing of the skin is disclosed in Patent Application FR-A-2 758 083 [1].

However, the use of these tensioning polymer systems sometimes produces a feeling of discomfort in some users, in particular those exhibiting delicate skin. In addition, the tensioning effect which they provide does not last for a very long time, in so far as the film formed on the skin has a tendency to crack under the effect of facial expressions. This is because these tensioning agents form a relatively rigid and not very deformable film on the skin.

The Applicant Company has found, surprisingly, that the use of a dispersion of a specific ethylenic polymer in combination with a tensioning agent in a cosmetic composition makes it possible to obtain, after topical application to wrinkled skin, films exhibiting a lasting tensioning effect, the said films being more deformable from a mechanical viewpoint.

In addition, it appeared to the Applicant Company that this dispersion of specific ethylenic polymers furthermore had the property of preventing the whitening of the skin resulting from the application to the latter of compositions comprising, as tensioning agents, colloidal dispersions of inorganic particles, in particular of silica.

### DESCRIPTION OF THE INVENTION

Thus, the invention relates, according to a first subject-matter, to a cosmetic process for softening the wrinkles of wrinkled skin comprising a stage consisting in applying, to said skin, a cosmetic composition, in particular an anti-wrinkle composition, comprising, in a physiologically acceptable medium suitable for topical application to the skin of the face:
from 0.1 to 20% by weight of at least one tensioning agent, with respect to the total weight of the composition, and
at least one dispersion in a liquid fatty phase of solid particles of a grafted ethylenic polymer.

The use of a dispersion as defined above in combination with a tensioning agent makes it possible to confer a persistent tensioning effect on the composition in which it is included, that is to say a tensioning effect which exhibits a degree of durability over time, the dispersion performing the role of reinforcing agent for the tensioning film. Tests targeted at demonstrating this property of persistence of such a combination are set out in the experimental part of this description. It is specified that this persistence is borne out in the context of this invention by the improvement in the mechanical properties of the tensioning film.

Before entering into further details in the description, the following definitions are provided.

The term “tensioning agent” is understood to mean, generally, according to the invention, any agent producing, at a concentration of 7% in water, a retraction of the isolated stratum corneum, measured with an extensometer, of more than 1% and preferably of more than 1.5% at 30° C. under a relative humidity of 40%.

The protocol for determining retraction of the stratum corneum is as follows:

The tensioning power of the tensioning agents described in the present document was measured with an extensometer.

The principle of the method consists in measuring the length of a test specimen of stratum corneum isolated from human skin originating from a surgical operation, before and after treatment with the test compositions.

To do this, the test specimen is placed between the two jaws of the device, one of which is fixed and the other of which is movable, in an atmosphere at 30° C. and 40% relative humidity. A pull is exerted on the test specimen and the curve of the force (in grams) as a function of the length (in millimetres) is recorded, the zero length corresponding to the contact between the two jaws of the device. The tangent to the curve in its linear region is subsequently plotted. The intersection of this tangent with the axis of the abscissae corresponds to the apparent length L₀ of the test specimen at zero force. The test specimen is subsequently relaxed and then 2 mg/cm² of the test composition (7% solution of the tensioning agent under consideration) are applied to the stratum corneum. After drying for 15 minutes, the above stages are again carried out in order to determine the length L₁ of the test specimen after treatment. The percentage of retraction is defined by: % retraction=100×(L₁−L₀)/L₀. In order to characterize a tensioning effect, this percentage has to be negative and the tensioning effect becomes greater as the absolute value of the percentage of retraction increases.

The said tensioning agent, in the context of this invention, can be chosen in particular from:
a) synthetic polymers;
b) polymers of natural origin;
c) mixed silicates;
d) wax microparticles;
e) colloidal particles of inorganic fillers;
and the mixtures of these.
   a) Synthetic Polymers

The synthetic polymers which can be used as tensioning agent can be chosen from:
polyurethane polymers and copolymers;
acrylic polymers and copolymers;
polymers of sulphoisophthalic acid;
grafted silicone polymers;
water-soluble or water-dispersible polymers comprising water-soluble or water-dispersible units and LCST units;
non-elastomeric and water-insoluble film-forming linear ethylenic block polymers exhibiting a dynamic storage modulus E′ at 1 Hz and at 22° C. of greater than 200 MPa;
a dispersion in a liquid fatty phase of solid particles of a grafted ethylenic polymer exhibiting a glass transition temperature of greater than 40° C.;
and mixtures of these.

The polyurethane copolymers, the acrylic copolymers and the other synthetic polymers according to the invention can be chosen in particular from polycondensates, hybrid polymers and interpenetrating polymer networks (IPNs).

The term “interpenetrating polymer network” (IPN), within the meaning of the present invention, is understood to mean a blend of two entangled polymers obtained by simultaneous polymerization and/or crosslinking of two types of monomers, the blend obtained having a single glass transition temperature.

Examples of IPNs suitable for use in the present invention, along with their process of preparation, are described in U.S. Pat. Nos. 6,139,322 [2] and 6,465,001 [3], for example.

Preferably, the IPN according to the invention comprises at least one polyacrylic polymer and, more preferably, it additionally comprises at least one polyurethane or one copolymer of vinylidene fluoride and of hexafluoropropylene.

According to a preferred embodiment, the IPN according to the invention comprises a polyurethane polymer and a polyacrylic polymer. Such IPNs are in particular those of the Hybridur series which are available commercially from Air Products.

A particularly preferred IPN is found in the form of an aqueous dispersion of particles having a mean size, by weight, of between 90 and 110 nm and a mean size, by number, of approximately 80 nm. This IPN preferably has a glass transition temperature Tg which ranges from approximately −60° C. to +100° C. An IPN of this type is sold in particular by Air Products under the trade name Hybridur X-01602. Another IPN suitable for use in the present invention is referenced Hybridur X18693-21.

Other IPNs suitable for use in the present invention comprise the IPNs composed of the blend of a polyurethane with a copolymer of vinylidene fluoride and of hexafluoropropylene. These IPNs can be prepared in particular as described in U.S. Pat. No. 5,349,003 [4]. In an alternative form, they are available commercially in the form of a colloidal dispersion in water, in a ratio of the fluorinated copolymer to the acrylic polymer of between 70:30 and 75:25, under the trade names Kynar RC-10,147 and Kynar RC-10,151 from Atofina.

Examples of grafted silicone polymers are shown in Application EP-1 038 519 [5], which is incorporated here by reference.

A grafted silicone polymer which can be used in the context of this invention comprises a polysiloxane portion and a portion composed of a non-silicone organic chain, one of the two portions constituting the main chain of the polymer and the other being grafted to the said main chain.

In a first alternative form where the said polymer is a polymer comprising a non-silicone organic backbone grafted by at least one monomer comprising a polysiloxane, it is preferably a grafted silicone copolymer comprising:
a) at least one lipophilic monomer (A) comprising ethylenic unsaturation which can be polymerized by the radical route;
b) at least one polar hydrophilic monomer (B) comprising ethylenic unsaturation which can be copolymerized with the monomer or monomers of the type (A) such that (A)+(B) varies from 99.99% to 50% by weight; and
c) from 0.01 to 50% by weight of at least one polysiloxane macromonomer (C) of following formula (I):
   X(Y)ₙSi(R)₃₋ₘZₘ (I)
   in which:
X denotes a group comprising ethylenic unsaturation which can be copolymerized with the monomers (A) and (B);
Y denotes a divalent bonding group;
R denotes a hydrogen, a hydroxyl group, a C₁-C₆ alkyl or alkylamino or alkoxy group or a C₆-C₁₂ aryl group;
Z denotes a polysiloxane unit having a number-average molecular weight of at least 50;
n is 0 or 1 and m is an integer ranging from 1 to 3; the percentages being calculated with respect to the total weight of the monomers (A), (B) and (C).

The monomer (A) can be chosen from the group consisting of n-butyl methacrylate, isobutyl methacrylate, tert-butyl acrylate, tert-butyl methacrylate, 2-ethylhexyl methacrylate, methyl methacrylate, 2-(N-methylperfluorooctylsulphonamido)-ethyl acrylate, 2-(N-butylperfluorooctylsulphonamido)-ethyl acrylate and their mixtures.

The monomer (B) can be chosen from the group consisting of acrylic acid, N,N-dimethyl-acrylamide, dimethylaminoethyl methacrylate, quaternized dimethylaminoethyl methacrylate, vinylpyrrolidone and their mixtures.

The polysiloxane macromonomer (C) has, for example, for formula the following formula (II): 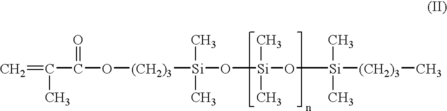
with n being an integer ranging from 1 to 700.

In a second preferred alternative form, the said polymer is a polymer comprising a polysiloxane backbone grafted by at least one non-silicone organic monomer.

This polymer advantageously results from the radical copolymerization between, on the one hand, at least one non-silicone anionic organic monomer exhibiting an ethylenic unsaturation (for example chosen from (meth)acrylic acids and their salts) and/or one non-silicone hydrophobic organic monomer exhibiting an ethylenic unsaturation (for example chosen from esters of (meth)acrylic acid with alkanols) and, on the other hand, a silicone exhibiting, in its chain, at least one functional group capable of reacting with the said ethylenic unsaturations of the said non-silicone monomers with the formation of a covalent bond.

In this case, the said silicone polymer preferably comprises, in its structure, the unit of following formula (III): 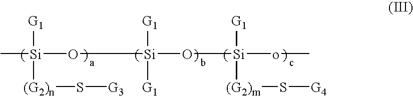
in which the radicals G₁, which are identical or different, represent a hydrogen or a C₁-C₁₀ alkyl radical or else a phenyl radical; the radicals G₂, which are identical or different, represent a C₁-C₁₀ alkylene group; G₃ represents a polymer residue resulting from the (homo)polymerization of at least one anionic monomer comprising ethylenic unsaturation; G4 represents a polymer residue resulting from the (homo)polymerization of at least one hydrophobic monomer comprising ethylenic unsaturation; m and n are, independently of one another, equal to 0 or 1; a is an integer ranging from 0 to.50; b is an integer which can be between 10 and 350; c is an integer ranging from 0 to 50; with the proviso that one of the parameters a and c is other than 0.

More preferably still, the said unit of formula (III) exhibits at least one and preferably all of the following characteristics:
the radicals G₁ denote a C₁-C₁₀ alkyl radical;
n is non-zero and the radicals G₂ represent a divalent C₁-C₃ radical;
G₃ represents a polymer radical resulting from the (homo)polymerization of at least one monomer of the carboxylic acid comprising ethylenic unsaturation type;
G₄ represents a polymer radical resulting from the (homo)polymerization of at least one monomer of the C₁-C₁₀ alkyl (meth)acrylate type.

The silicone polymer can thus be a polydimethylsiloxane onto which are grafted, via a connecting link of thiopropylene type, mixed polymer units of the poly((meth)acrylic acid) type and of the poly(alkyl (meth)acrylate) type.

A particularly preferred example of grafted silicone polymer is polysilicone-8 (known under the CTFA abbreviation), which is a polydimethylsiloxane onto which are grafted, via a connecting link of thiopropylene type, mixed polymer units of the poly((meth)acrylic acid) type and of the poly(alkyl (meth)acrylate) type. A polymer of this type is available in particular under the trade name VS 80 (at 10% in water) or LO 21 (in pulverulent form) from 3M. It is a polydimethylsiloxane copolymer comprising propylthio, methyl acrylate, methyl methacrylate and methacrylic acid groups.

The abovementioned synthetic polymers can be provided in the latex form. Mention may in particular be made, as appropriate latex which can be used according to the invention as tensioning agent, of polyester/polyurethane and polyether/polyurethane dispersions, such as those sold under the names “Avalure UR410 and UR460” by Noveon, and under the names “Neorez R974”, “Neorez R981”, “Neorez R970”, and acrylic copolymer dispersions, such as those sold under the name “Neocryl XK-90” by Avecia.

Use may also be made, according to the invention, of water-soluble or water-dispersible polymers comprising water-soluble or water-dispersible units and comprising LCST units, the said LCST units exhibiting in particular a phase-separation temperature in water of 5 to 40° C. at a concentration by weight of 1%. This type of polymer is more fully described in Patent Application FR 2 819 429 [6].

Other synthetic polymers which can be used as tensioning agent in the context of the invention are non-elastomeric and water-insoluble film-forming linear ethylenic block polymers exhibiting a dynamic storage modulus E′ at 1 Hz and at 22° C. of greater than 200 MPa, such as those described in Application FR 03/11346 [7].

Finally, tensioning agents which can be used in the context of the invention can be a dispersion in a liquid fatty phase of solid particles of a grafted ethylenic polymer exhibiting a glass transition temperature of greater than 40° C.

It is understood that this dispersion as defined in the above paragraph differs from that incorporated in the composition for the purpose of enhancing the persistence of the tensioning effect, the latter advantageously having, in this case, a glass transition temperature of less than or equal to 40° C. Mention may be made, as example of tensioning agent of this type, of a dispersion obtained by polymerization in isododecane of methyl acrylate, of acrylic acid and of the macromonomer polyethylene/polybutylene methacrylate (such as Kraton L-1253).

b) Polymers of Natural Origin

The polymers of natural origin which can be used as tensioning agent can be chosen from:
plant proteins and plant protein hydrolysates;
polysaccharides of plant origin in the form of microgels, such as starch;
latexes of plant origin,
and mixtures of these.

Examples of plant proteins and plant protein hydrolysates which can be used as tensioning agents according to the invention are composed of proteins and protein hydrolysates of maize, rye, wheat, buckwheat, sesame, spelt, peas, broad beans, lentils, soybeans and lupin.

Polysaccharides suitable for the formulation of the compositions according to the invention are all polysaccharides of natural origin capable of forming thermally reversible or crosslinked gels, and also solutions. The term “thermally reversible” is understood to mean the fact that the gel state of these polymer solutions is obtained reversibly once the solution has cooled below the gelling temperature characteristic of the polysaccharide used.

A first family of polysaccharides of natural origin which can be used in the present invention is composed of carrageenans and very particularly κ-carrageenan and ι-carrageenan. These are. linear polysaccharides present in certain red algae. They are composed of alternating β-1,3- and α-1,4-galactose residues, of numerous galactose residues which can be sulphated. This family of polysaccharides is described in the work “Food Gels” edited by Peter Harris, Elsevier, 1989, Chap. 3 [8]. Another family of polysaccharides which can be used is composed of agars. These are also polymers extracted from red algae and they are composed of alternating 1,4-L-galactose and 1,3-D-galactose residues. This family of polysaccharides is also described in Chapter 1 of the work “Food Gels” [9] mentioned above. A third family of polysaccharides is composed of polysaccharides of bacterial origin, known as gellans. These are polysaccharides composed of an alternation of glucose, glucuronic acid and rhamnose residues. Gellans are described in particular in Chapter 6 of the work “Food Gels” [10] mentioned above. In the case of the polysaccharides which form gels of crosslinked type, in particular induced by addition of salts, mention will be made of the polysaccharides belonging to the family of the alginates and pectins.

Mention may also be made of pullulans and their derivatives, and also of mixtures of polymers with opposite charges which form complexes via electrostatic interactions.

The tensioning polysaccharides are present in the form of microgels as described in FR 2 829 025 [11].

A particularly advantageous category of polysaccharides which can be used according to the invention is composed of starch and its derivatives.

Starch is a natural product well known to a person skilled in the art. It consists of a polymer or a mixture of polymers which are linear or branched and which are composed of α-D-glucopyranosyl units. Starch is described in particular in “Kirk-Othmer Encyclopedia of Chemical Technology, 3rd edition, Volume 21, pp. 492-507, Wiley Interscience, 1983” [12].

The starch employed according to the present invention can be of any origin: rice, maize, potato, manioc, peas, wheat, oats, and the like. It can be natural or optionally modified by a treatment of crosslinking, acetylation or oxidation type. It can optionally be grafted.

c) Mixed Silicates

Another category of tensioning agents which can be used according to the invention is composed of mixed silicates. This expression is understood to mean all silicates of natural or synthetic origin which include several types of cations chosen from alkali metals (for example, Na, Li, K) or alkaline earth metals (for example, Be, Mg, Ca) and transition metals.

Use is preferably made of phyllosilicates, namely silicates having a structure in which the SiO₄ tetrahedra are arranged in sheets between which the metal cations are found enclosed.

A family of silicates which is particularly preferred as tensioning agents is that of the laponites. Laponites are magnesium, lithium and sodium silicates having a layered structure similar to that of montmorillonites. Laponite is the synthetic form of the natural mineral referred to as “hectorite”. Use may be made, for example, of the laponite sold under the name Laponite XLS or Laponite XLG by Rockwood.

d) Wax Microdispersions

Yet another category of tensioning agents which can be used in the present invention is composed of microdispersions of wax particles. They are dispersions of particles having a diameter generally of less than 5 μm or better still of less than 0.5 μm and composed essentially of a wax or of a mixture of waxes chosen, for example, from carnauba, candelilla or alfa waxes. The melting point of the wax or of the mixture of waxes is preferably between 50° C. and 150° C.

e) Colloidal Particles of Inorganic Fillers

In another alternative form, use may be made, as tensioning agent according to the invention, of colloidal particles of inorganic fillers. The term “colloidal particles” is understood to mean colloidal particles in dispersion in an aqueous, aqueous/alcoholic or alcoholic medium having a number-average diameter of between 0.1 and 100 nm, preferably between 3 and 30 nm.

These particles are provided in the form of aqueous dispersions and do not have any tensioning property in the water, the alcohol, the oil and any other solvent. At a concentration of greater than or equal to 15% by weight in water, the viscosity of the solutions thus obtained is less than 0.05 Pa·s for a shear rate of 10 s⁻¹. The viscosity measurements are carried out at 25° C. using a Rheostress RS150 rheometer from Haake in cone/plate configuration, the dimensions of the measuring cone being: diameter: 60 mm and angle: 2°.

Examples of inorganic fillers comprise: silica, cerium oxide, zirconium oxide, alumina, calcium carbonate, barium sulphate, calcium sulphate, zinc oxide and titanium dioxide. Silica is a particularly preferred inorganic filler. Colloidal silica particles are available in particular in the form of an aqueous colloidal silica dispersion from Catalysts & Chemicals under the trade names Cosmo S-40 and Cosmo S-50.

A specific example of colloidal particles of inorganic fillers can be silica/alumina composite colloidal particles. The term “silica/alumina composite” is understood to mean silica particles in which the aluminium atoms have been partially substituted by silica atoms.

At a pH of 7, the silica/alumina composite colloidal particles according to the invention have a zeta potential of less than −20 mV and preferably of less than −25 mV. The measurements are carried out at 25° C. using a Delsa 440SX device from Coulter Scientific Instrument.

Mention may be made, as silica/alumina composite colloidal particles which can be used in the compositions according to the invention, for example, of those sold by Grace under the names of. Ludox AM, Ludox HSA and Ludox TMA.

Whatever the nature of the tensioning agent present in the composition, it is present in the composition at a content ranging from 0.1 to 20% by weight of the total weight of the composition, preferably from 1 to 10%.

As mentioned above, the composition comprises a dispersion of solid particles, in a liquid fatty phase, of a grafted ethylenic polymer.

In order to form such a dispersion of solid particles, the polymers result from a careful choice of monomers constituting the main chain (for example, composed predominantly of short alkyl acrylate or methacrylate monomers) and of macromonomers constituting the grafts (for example present in a proportion representing less than 20% of the weight of the polymer).

Such a dispersion, in combination with a tensioning agent, confers, on the composition in which it is incorporated, a persistent tensioning effect by virtue of its ability to strengthen the tensioning film while contributing properties of flexibility to it. This persistence is quantified according to the invention in particular by the measurement of the improvement in the mechanical properties of the tensioning film (more particularly by the measurement of the improvement in the breaking strength), as will be explained in the protocol appearing in the experimental part of this description.

This dispersion is advantageously present in the composition at a content ranging, as active material, from 0.01 to 20% of the total weight of the composition, preferably from 1 to 10%, the said dispersion preferably being present at the most in an amount equal to that of the tensioning agent.

According to a specific embodiment of the invention, the grafted ethylenic polymer forming a dispersion of solid particles in a liquid fatty phase (and fulfilling the role of agent which strengthens the tensioning effect) comprises a backbone which is insoluble in the said liquid fatty phase and a part which is soluble in the said liquid fatty phase composed of side chains covalently bonded to the said backbone.

In particular, the grafted ethylenic polymer can be a grafted acrylic polymer.

Such a grafted acrylic polymer can in particular be capable of being obtained by radical polymerization in the said liquid fatty phase:
of at least one acrylic monomer and optionally of at least one additional non-acrylic vinyl monomer, in order to form the said insoluble backbone; and
of at least one macromonomer comprising a polymerizable end group, in order to form the side chains, the said macromonomer having a weight-average molecular weight of greater than or equal to 200 and the content of polymerized macromonomer representing from 0.05 to 20% by weight of the polymer.

The weight-average molecular weight of the polymer can range, according to the invention, from 10 000 to 300 000, preferably from 20 000 to 200 000 and better still from 25 000 to 150 000.

The choice of the monomers constituting the backbone of the polymer and of the macromonomers, as well as the weight-average molecular weight of the polymer, of the side chains, and also the proportion of the side chains, can be made according to the liquid fatty phase chosen, so as to obtain a dispersion of solid particles of polymers in the said phase, and advantageously a stable dispersion, it being possible for this choice to be made by a person skilled in the art.

The term “stable dispersion” is understood to mean a dispersion which is not capable of forming a solid deposit or of solid/liquid phase separation, in particular after centrifuging, for example, at 4000 revolutions/minute for 15 minutes.

The grafted acrylic polymer can be a random polymer.

Thus, the grafted.acrylic polymer comprises a backbone (or main chain) composed of a sequence of acrylic units resulting from the polymerization in particular of one or more acrylic monomers and side chains (or grafts) resulting from the reaction of the macromonomers, the said side chains being covalently bonded to the said main chain.

The backbone (or main chain) is insoluble in the liquid fatty phase under consideration, whereas the side chains (or grafts) are soluble in the said phase.

By virtue of the abovementioned characteristics, in a given liquid phase, the polymers have the ability to withdraw into themselves, thus forming particles of substantially spherical shape, with the side chains opened out on the perimeter of these particles, which side chains ensure the stability of these particles. Such particles, resulting from the characteristics of the grafted polymer, have the distinguishing feature of not agglomerating in the said phase and thus of self-stabilizing and of forming a particularly stable dispersion of polymer particles.

In particular, the polymers of the invention can form nanometric particles with a mean size ranging from 10 to 400 nm, preferably from 20 to 200 nm, in the liquid fatty phase under consideration.

Due to this very small size, the dispersed grafted polymer particles. are particularly stable and thus not very susceptible to forming agglomerates.

The grafted polymer dispersion can thus be a dispersion which is stable in the phase under consideration and does not form sediments when it is placed for a prolonged period of time (for example 24 hours) at ambient temperature (25° C.).

The sizes of the particles can be measured by various techniques. Mention may in particular be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

Preferably, the sizes and size distributions of the particles of the compositions according to the invention are measured by static light scattering using a commercial particle sizer of MasterSizer 2000 type from Malvern. The data are processed on the basis of the Mie scattering theory. This theory, exact for isotropic particles, makes it possible to determine, in the case of non-spherical particles, an “effective” particle diameter. This theory is described in particular in the work by Van de Hulst, H. C., “Light Scattering by Small Particles”, Chapters 9 and 10, Wiley, New York, 1957 [13].

The composition is characterized by its mean “effective” diameter by volume D[4,3], defined in the following way:

$$D [ 4 , 3 ] = \frac{{\sum }_{i} {V}_{i} \cdot {d}_{i}}{{\sum }_{i} {V}_{i}}$$

where Vᵢ represents the volume of the particles with an effective diameter dᵢ. This parameter is described in particular in the technical documentation of the particle sizer.

The measurements are carried out at 25° C. on a dilute dispersion of particles obtained from the composition in the following way:
1) dilution by a factor of 100 with water,
2) homogenization of the solution,
3) standing of the solution for 18 hours,
4) recovery of the off-white homogeneous supernatant.

The “effective” diameter is obtained by taking a refractive index of 1.33 for the water and a mean refractive index of 1.42 for the particles.

Advantageously, the acrylic monomers represent from 50 to 100% by weight, preferably from 55 to 100% by weight (in particular from 55 to 95% by weight), preferentially from 60 to 100% by weight (in particular from 60 to 90% by weight), of the acrylic monomers+optional non-acrylic vinyl monomers mixture.

Preferably, the acrylic monomers are chosen from monomers, the homopolymer of which is insoluble in the liquid fatty phase under consideration, that is to say that the homopolymer is in the solid (or undissolved) form at a concentration of greater than or equal to 5% by weight at ambient temperature (20° C.) in the said liquid fatty phase, the said % being expressed with respect to the total weight of the mixture composed of the homopolymer and the liquid fatty phase under consideration.

The term “acrylic monomer” is understood to mean, in the present application, monomers chosen from (meth)acrylic acid, esters of (meth)acrylic acid (also known as (meth)acrylates), amides of (meth)acrylic acid (also known as (meth)acrylamides).

Mention may be made, as acrylic monomer capable of being employed to form the insoluble backbone of the polymer, alone or as a mixture, of the following monomers:
(i) (meth)acrylates of following formula (IV): 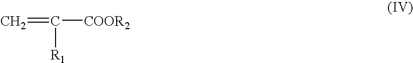
   in which:
R₁ denotes a hydrogen atom or a methyl group;
R₂ represents a group chosen from:
   a linear or branched alkyl group comprising from 1 to 6 carbon atoms, it being possible for the said group to comprise, in its chain, one or more heteroatoms chosen from O, N and S; and/or it being possible for the said group to comprise one or more substituents chosen from —OH, halogen atoms (F, Cl, Br, I) and —NR′R″ with R′ and R″, which are identical or different, being chosen from linear or branched C₁-C₄ alkyl groups; and/or it being possible for the said group to be substituted by at least one polyoxyalkylene group, in particular a C₁-C₄ alkylene group, especially a polyoxyethylene and/or a polyoxypropylene, the said polyoxyalkylene group being composed of the repetition of 5 to 30 oxyalkylene units;
   a cyclic alkyl group comprising from 3 to 6 carbon atoms, it being possible for the said group to comprise one or more heteroatoms chosen from O, N and S and/or it being possible for the said group to comprise one or more substituents chosen from OH and halogen atoms (F, Cl, Br, I);
mention may be made, as examples of R₂, of the methyl, ethyl, propyl, butyl, isobutyl, methoxyethyl, ethoxyethyl, methoxy(polyoxyethylene 30 OE) (OE meaning oxyethylene), trifluoroethyl, 2-hydroxyethyl, 2-hydroxypropyl, dimethylaminoethyl, diethylaminoethyl or dimethylaminopropyl group;
   (ii) (meth)acrylamides of following formula (V): 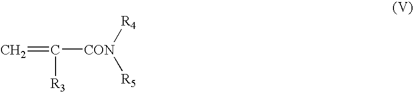
      in which:
   R₃ denotes a hydrogen atom or a methyl group;
   R₄ and R₅, which are identical or different, represent a hydrogen atom or a linear or branched alkyl group comprising from 1 to 6 carbon atoms which can comprise one or more substituents chosen from —OH, halogen atoms (F, Cl, Br, I) and —NR′R″ with R′ and R″, which are identical or different, being chosen from linear or branched C₁-C₄ alkyl groups; or
   R₄ represents a hydrogen atom and R₅ represents a 1,1-dimethyl-3-oxobutyl group;
mention may be made, as examples of alkyl groups which R₄ and R₅ can constitute, of the n-butyl, t-butyl, n-propyl, dimethylaminoethyl, diethylaminoethyl and dimethylaminopropyl groups;
   (iii) (meth)acrylic monomers comprising at least one carboxylic, phosphoric or sulphonic acid functional group, such as acrylic acid, methacrylic acid or acrylamidomethylpropanesulphonic acid;
      it being possible for the said monomers to be present in the form of salts.

Mention may very particularly be made, among these acrylic monomers, of methyl (meth)acrylate, ethyl (meth)acrylate, propyl (meth)acrylate, butyl (meth)acrylate, isobutyl (meth)acrylate, methoxyethyl (meth)acrylate, ethoxyethyl (meth)acrylate, trifluoro-ethyl methacrylate, dimethylaminoethyl methacrylate, diethylaminoethyl methacrylate, 2-hydroxypropyl methacrylate, 2-hydroxyethyl methacrylate, 2-hydroxy-propyl acrylate, 2-hydroxyethyl acrylate, dimethyl-aminopropylmethacrylamide, (meth)acrylic acid and their salts.

Preferably, the acrylic monomers are chosen from methyl acrylate, methoxyethyl acrylate, methyl methacrylate, 2-hydroxyethyl methacrylate, (meth)-acrylic acid, dimethylaminoethyl methacrylate and their mixtures.

Mention may be made, among the. additional non-acrylic vinyl monomers, of:
vinyl esters of following formula (VI):
   R₆—COO—CH═CH₂ (VI)
   in which R₆ represents a linear or branched alkyl group comprising from 1 to 6 carbon atoms or a cyclic alkyl group comprising from 3 to 6 carbon atoms and/or an aromatic group, for example of benzene, anthracene and naphthalene type;
non-acrylic vinyl monomers comprising at least one carboxylic, phosphoric or sulphonic acid functional group, such as crotonic acid, maleic anhydride, itaconic acid, fumaric acid, maleic acid, styrene-sulphonic acid, vinylbenzoic acid, vinylphosphoric acid and the salts of these;
non-acrylic vinyl monomers comprising at least one tertiary amine functional group, such as 2-vinyl-pyridine or 4-vinylpyridine;
and their mixtures.

Mention may be made, among the salts, of those obtained by neutralization of the acid groups using inorganic bases, such as sodium hydroxide, potassium hydroxide or ammonium hydroxide, or organic bases of alkanolamine type, such as monoethanolamine, diethanolamine, triethanolamine or 2-methyl-2-amino-1-propanol.

Mention may also be made of the salts formed by neutralization, if appropriate, of the tertiary amine units, for example using an inorganic or organic acid. Mention may be made, among inorganic acids, of sulphuric acid, hydrochloric acid, hydrobromic acid, hydriodic acid, phosphoric acid or boric acid. Mention may be made, among organic acids, of acids comprising one or more carboxyl, sulpho or phosphono groups. They can be linear, branched or cyclic aliphatic acids or else aromatic acids. These acids can additionally comprise one or more heteroatoms chosen from O and N, for example in the form of hydroxyl groups. Mention may in particular be made of acetic acid or propionic acid, terephthalic acid, and also citric acid and tartaric acid.

The grafted polymer may not comprise additional non-acrylic vinyl monomers as described above. In this embodiment, the insoluble backbone of the grafted polymer is formed solely of acrylic monomers as described above.

It is understood that these non-polymerized acrylic monomers may be soluble in the medium under consideration but the polymer formed by polymerization of these monomers is insoluble in the liquid fatty phase under consideration.

According to the invention, the grafted acrylic polymer comprises side chains resulting from the polymerization of macromonomers having a polymerizable end group.

The term “macromonomer having a polymerizable end group” is understood to mean any oligomer comprising, on just one of its ends, a polymerizable end group capable of reacting during the polymerization reaction with the acrylic monomers and optionally the additional non-acrylic vinyl monomers constituting the backbone. The macromonomer makes it possible to form the side chains of the grafted acrylic polymer. The polymerizable group of the macromonomer can advantageously be a group comprising ethylenic unsaturation capable of polymerizing by the radical route with the monomers constituting the backbone.

The macromonomers comprise, at one of the ends of the chain, a polymerizable end group capable of reacting during the polymerization with the acrylic monomers and optionally the additional vinyl monomers to form the side chains of the grafted polymer. The said polymerizable end group can in particular be a vinyl or (meth)acrylate (or (meth)acryloyloxy) group and preferable a (meth)acrylate group.

The macromonomers are preferably chosen from macromonomers, the homopolymer of which has a glass transition temperature (Tg) of less than or equal to 25° C., in particular ranging from −100° C. to 25° C., preferably ranging from −80° C. to 0C.

Preferably, the macromonomer is chosen from macromonomers, the homopolymer of which is soluble in the liquid fatty phase under consideration, that is to say completely dissolved at a concentration of greater than or equal to 5% by weight and at ambient temperature (20° C.) in the said liquid fatty phase, the said % being expressed with respect to the total weight of the mixture composed of the homopolymer and of the liquid fatty phase under consideration.

Preferably, the polymerized macromonomer (constituting the side chains of the grafted polymer) represents from 0.1 to 15% by weight of the total weight of the polymer, preferably from 0.2 to 10% by weight and more preferably from 0.3 to 8% by weight.

The macromonomers generally have a weight-average molecular weight (Mw) of greater than 200, preferably of greater than 300, better still of greater than 500 and even better still of greater than 600. They preferably have a weight-average molecular weight ranging from 200 to 100 000, preferably ranging from 500 to 50 000, preferentially ranging from 800 to 20 000, more preferentially ranging from 800 to 10 000 and more preferentially still ranging from 800 to 6000.

In the present application, the weight-average molar masses (Mw) and the number-average molar masses (Mn) are determined by gel permeation liquid chromatography (solvent THF, calibration curve drawn up with linear polystyrene standards, refractometric detector).

As mentioned above, the strengthening agents of the invention constitute a dispersion of solid particles of a grafted ethylenic polymer in a liquid fatty phase.

This liquid fatty phase is composed mainly of one or more liquid organic compounds which will be defined below.

The term “liquid organic compound” is understood to mean a non-aqueous compound which is in the liquid state at ambient temperature (25° C.), that is to say which flows under the effect of its own weight.

Mention may be made, among liquid organic compounds which can be present in the liquid fatty phase, of:
liquid organic compounds having an overall solubility parameter according to the Hansen solubility space of less than or equal to 18 (MPa)^{1/2}, preferably of less than or equal to 17 (MPa)^{1/2};
monoalcohols having an overall solubility parameter according to the Hansen solubility space of less than or equal to 20 (MPa)^{1/2}; and
their mixtures.

The overall solubility parameter δ according to the Hansen solubility space is defined in the article “Solubility parameter values” by Eric A. Grulke in the work “Polymer Handbook”, 3rd edition, Chapter VII, pp. 519-559 [14], by the relationship:
δ=(d_{D}²+dₚ²+d_{H}²)^{1/2}
in which
d_{D} characterizes the London dispersion forces resulting from the formation of dipoles induced during molecular impacts,
dₚ characterizes the forces of Debye interactions between permanent dipoles, and
d_{H} characterizes the forces of specific interactions (hydrogen bond, acid/base or donor/acceptor type, and the like).

The definition of the solvents in the solubility space according to Hansen is described in the paper by C. M. Hansen, “The three-dimensional solubility parameters”, J. Paint Technol., 39, 105 (1967) [15].

According to a first alternative, the liquid fatty phase can be a non-silicone fatty phase.

The term “non-silicone liquid fatty phase” is understood to mean a fatty phase comprising one or more non-silicone liquid organic compounds chosen from:
non-silicone liquid organic compounds having an overall solubility parameter according to the Hansen solubility space of less than or equal to 18 (MPa)^{1/2}, preferably of less than or equal to 17 (MPa)^{1/2};
liquid monoalcohols having an overall solubility parameter according to the Hansen solubility space of less than or equal to 20 (MPa)^{1/2}; and
their mixtures,
   the said non-silicone compounds being present as the predominant component in the liquid fatty phase, that is to say at at least 50% by weight, in particular from 50 to 100% by weight, for example from 60 to 99% by weight, or even from 65 to 95% by weight, with respect to the total weight of the liquid fatty phase.

The said non-silicone liquid fatty phase can thus optionally comprise silicone liquid organic compounds or silicone oils, such as those mentioned below, which can be present in an amount of less than 50% by weight, in particular ranging from 0.1 to 40% by weight, indeed even ranging from 1 to 35% by weight, or even ranging from 5 to 30% by weight, with respect to the total weight of the liquid fatty phase.

According to a specific embodiment of the invention, the non-silicone liquid fatty phase does not comprise silicone liquid organic compounds or silicone oils.

The non-silicone liquid compounds having an overall solubility parameter according to the Hansen solubility space of less than 18 (MPa)^{1/2}, preferably of less than or equal to 17 (MPa)^{1/2} can be chosen from:
optionally branched, carbon, hydrocarbon or fluorinated, natural or synthetic oils, alone or as a mixture;
optionally volatile, linear, branched and/or cyclic alkanes;
esters and in particular linear, branched or cyclic esters having at least 6 carbon atoms, in particular having from 6 to 30 carbon atoms;
ethers and in particular ethers having at least 6 carbon atoms, in particular having from 6 to 30 carbon atoms;
ketones and in particular ketones having at least 6 carbon atoms, in particular having from 6 to 30 carbon atoms.

Mention may be made, among the oils, of vegetable oils formed by esters of fatty acids and of polyols, in particular triglycerides, such as sunflower, sesame or rapeseed oil.

Mention may also be made of linear, branched and/or cyclic alkanes which are optionally volatile and in particular liquid paraffin, liquid petrolatum, or hydrogenated polyisobutylene, isododecane, or even the “Isopars”, volatile isoparaffins.

The term “liquid monoalcohols having an overall solubility parameter according to the Hansen solubility space of less than or equal to 20 (MPa)^{1/2}” is understood to mean saturated or unsaturated liquid aliphatic fatty monoalcohols having at least 6 carbon atoms, for example from 6 to 30 carbon atoms, the hydrocarbon chain not comprising a substituent group. Mention may be made, as monoalcohols according to the invention, of oleyl alcohol, decanol and linoleyl alcohol.

In such a liquid fatty phase, the polymers forming a dispersion can, according to one embodiment of the invention, be grafted acrylic polymers as defined above.

Preferably, the macromonomers present in such a grafted polymer are advantageously carbon macromonomers.

The term “carbon macromonomer” is understood to mean a non-silicone macromonomer and in particular an oligomeric macromonomer obtained by polymerization of non-silicone monomer(s) comprising ethylenic unsaturation and mainly by polymerization of acrylic and/or non-acrylic vinyl monomers.

Mention may in particular be made, as carbon macromonomers, of:
(i) linear or branched C₈-C₂₂ alkyl (meth)acrylate homopolymers and copolymers exhibiting a polymerizable end group chosen from vinyl or (meth)acrylate groups, among which may in particular be mentioned: poly(2-ethylhexyl acrylate) macromonomers comprising a mono(meth)acrylate end; poly(dodecyl acrylate) or poly(dodecyl methacrylate) macromonomers comprising a mono(meth)acrylate end; poly(stearyl acrylate) or poly(stearyl methacrylate) macromonomers comprising a mono(meth)acrylate end.

Such macromonomers are described in particular in Patents EP 895 467 [16] and EP 96 459 [17] and in the paper by Gillman K. F., Polymer Letters, Vol. 5, pages 477-481 (1967) [18].

Mention may in particular be made of the macromonomers based on poly(2-ethylhexyl acrylate) or on poly(dodecyl acrylate) comprising a mono(meth)-acrylate end.
(ii) polyolefins having an end group comprising ethylenic unsaturation, in particular having a (meth)acrylate end group. Mention may in particular be made, as examples of such polyolefins, of the following macromonomers, it being understood that they have a (meth)acrylate end group: polyethylene macromonomers, polypropylene macromonomers, polyethylene/polypropylene copolymer macromonomers, polyethylene/polybutylene copolymer macromonomers, polyisobutylene macromonomers; polybutadiene macromonomers, polyisoprene macromonomers, polybutadiene macromonomers, poly(ethylene /butylene)-polyisoprene macromonomers.

Such macromonomers are described in particular in U.S. Pat. No. 5,625,005 [19], which mentions polyethylene/polybutylene and polyethylene/polypropylene macromonomers comprising a (meth)acrylate reactive end group.

Mention may in particular be made of poly(ethylene/butylene) methacrylate, such as that sold under the name Kraton Liquid L-1253 by Kraton Polymers.

A composition which is particularly effective in the context of the invention is a composition in which the dispersion (fulfilling the role of strengthening agent) is a dispersion obtained by polymerization of methyl acrylate and of the macromonomer polyethylene/polybutylene methacrylate (in particular Kraton L-1253) in isododecane and the tensioning agent is preferably a colloidal silica dispersion.

Another composition which is particularly effective in the context of the invention is a composition in which the dispersion (fulfilling the role of strengthening agent) is a dispersion obtained by polymerization of methyl acrylate and of the macromonomer polyethylene/polybutylene methacrylate (in particular Kraton L-1253) in isododecane and the tensioning agent is preferably a dispersion obtained by polymerization in isododecane of methyl acrylate, of acrylic acid and of the macromonomer polyethylene/polybutylene methacrylate (in particular Kraton L-1253).

According to a second alternative, the liquid fatty phase can be a silicone fatty phase.

The term “silicone liquid fatty phase” is understood to mean a fatty phase comprising one or more silicone liquid organic compounds chosen from silicone liquid organic compounds having an overall solubility parameter according to the Hansen solubility space of less than or equal to 17 (MPa)^{1/2}, the said silicone compounds being present as the predominant component in the liquid fatty phase, that is to say at at least 50% by weight, in particular from 50 to 100% by weight, for example from 60 to 99% by weight, or even from 65 to 95% by weight, with respect to the total weight of the liquid fatty phase.

The said silicone liquid fatty phase can thus optionally comprise non-silicone liquid organic compounds or non-silicone oils as described above, which can be present in an amount of less than 50% by weight, in particular ranging from 0.1 to 40% by weight, indeed even ranging from 1 to 35% by weight, or even ranging from 5 to 30% by weight, with respect to the total weight of the liquid fatty phase.

According to a specific embodiment of the invention, the silicone liquid fatty phase does not comprise non-silicone liquid organic compounds.

Among the silicone compounds corresponding to the above definition, mention may be made of silicone oils, such as polydimethylsiloxanes and polymethylphenylsiloxanes, optionally substituted by optionally fluorinated aliphatic and/or aromatic groups or by functional groups, such as hydroxyl, thiol and/or amine groups, and volatile silicone oils, in particular cyclic oils.

Mention may in particular be made of optionally branched, volatile and/or non-volatile silicone oils.

The term “volatile oil” is understood to mean an oil capable of evaporating from the skin or lips in less than one hour, having in particular a vapour pressure, at ambient temperature and atmospheric pressure, ranging from 10^{31 3} to 300 mmHg (0.13 Pa to 40 000 Pa).

Mention may be made, as volatile silicone oil which can be used in the invention, of linear or cyclic silicone oils having from 2 to 7 silicon atoms, these silicone oils optionally comprising alkyl or alkoxy groups having from 1 to 10 carbon atoms. Mention may in particular be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane and their mixtures.

Mention may be made, as non-volatile silicone oil, of non-volatile polydialkylsiloxanes, such as non-volatile polydimethylsiloxanes (PDMS); polydimethylsiloxanes comprising pending alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or polymethylphenylsiloxanes; polysiloxanes modified with fatty acids (in particular C₈-C₂₀ fatty acids), fatty alcohols (in particular C₈-C₂₀ fatty alcohols) or polyoxyalkylenes (in particular polyoxyethylene and/or polyoxypropylene); aminated polysiloxanes; polysiloxanes comprising hydroxyl groups; fluorinated polysiloxanes comprising a pending fluorinated group or a fluorinated group at the end of the silicone chain having from 1 to 12 carbon atoms, all or part of the hydrogens of which are substituted by fluorine atoms; and their mixtures.

In such a liquid fatty phase, the polymers forming a dispersion can, according to one embodiment of the invention, be grafted acrylic polymers such as those defined above.

Preferably, the macromonomers present in such a grafted acrylic polymer are advantageously silicone macromonomers.

The term “silicone macromonomer” is understood to mean an organopolysiloxane macromonomer and in particular a polydimethylsiloxane macromonomer.

Polymers forming, in a silicone liquid phase, a dispersion which is effective as strengthening agent are those chosen from the polymers capable of being obtained by radical polymerization in the said phase:
of a main (that is to say, representing more than 50% by weight) acrylic monomer chosen from C₁-C₃ alkyl (meth)acrylates, alone or as a mixture, and optionally of one or more additional (that is to say, representing less than 50% by weight) acrylic monomers chosen from acrylic acid, methacrylic acid and the alkyl (meth)acrylates of formula (VII) defined below, and their salts, in order to form the said insoluble backbone;
and of at least one silicone macromonomer comprising a polymerizable end group as defined above.

Use may be made, as main acrylic monomer, of methyl acrylate, methyl methacrylate, ethyl acrylate, ethyl methacrylate, n-propyl acrylate, n-propyl methacrylate, isopropyl acrylate and isopropyl methacrylate, and their mixtures. Preference is very particularly given to methyl acrylate, methyl methacrylate and ethyl methacrylate.

The additional acrylic monomers can be chosen from:
(meth)acrylic acid and its salts,
the (meth)acrylates of formula (VII) and their salts: 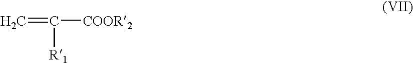
   in which:
R′₁ denotes a hydrogen atom or a methyl group;
R′₂ represents:
   a linear or branched alkyl group comprising from 1 to 6 carbon atoms, the said group comprising, in its chain, one or more oxygen atoms and/or comprising one or more substituents chosen from —OH, halogen atoms (F, Cl, Br, I) and —NR′R″ with R′ and R″, which are identical or different, being chosen from linear or branched C₁-C₃ alkyl groups; or
   a cyclic alkyl group comprising from 3 to 6 carbon atoms, it being possible for the said group to comprise, in its chain, one or more oxygen atoms and/or it being possible for the said group to comprise one or more substituents chosen from OH and halogen atoms (F, Cl, Br, I); or
   their mixtures.

Mention may be made, as examples of R′₂, of the methoxyethyl, ethoxyethyl, trifluoroethyl, 2-hydroxyethyl, 2-hydroxypropyl, dimethylaminoethyl, diethylaminoethyl or dimethylaminopropyl group.

Mention may very particularly be made, among these additional acrylic monomers, of (meth)acrylic acid, methoxyethyl (meth)acrylate, ethoxyethyl (meth)acrylate, trifluoroethyl methacrylate, dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate, their salts and their mixtures.

Preference is very particularly. given to acrylic acid or methacrylic acid.

Mention may in particular be made, as silicone macromonomers, of polydimethylsiloxanes comprising a mono(meth)acrylate end group and in particular of those of following formula (VIII): 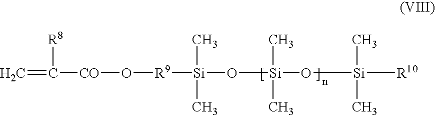
in which:
R⁸ represents a hydrogen atom or a methyl group;
R⁹ represents a divalent hydrocarbon group having from 1 to 10 carbon atoms and optionally comprising one or two ether —O— bonds;
R¹⁰ represents an alkyl group having from 1 to 10 carbon atoms, in particular from 2 to 8 carbon atoms;
n represents an integer ranging from 1 to 300, preferably ranging from 3 to 200 and preferentially ranging from 5 to 100.

Mention may in particular be made of polydimethylsiloxanes comprising a mono(meth)acrylate end group and in particular of monomethacryloyloxy -propylpolydimethylsiloxanes, such as those sold under the name PS560-K6 by United Chemical Technologies Inc. (UCT) or under the name MCR-M17 by Gelest Inc.

A composition which is particularly effective in the context of the invention is a composition in which the dispersion (fulfilling the role of strengthening agent) is a dispersion obtained by polymerization of methyl acrylate and of the macromonomer monomethacryloyloxypropylpolydimethylsiloxane in cyclopentadimethylsiloxane and the tensioning agent is, preferably, a colloidal silica dispersion.

Another composition which is effective in the context of the invention is a composition in which the dispersion (fulfilling the role of strengthening agent) is a dispersion obtained by polymerization of methyl acrylate and of the macromonomer monomethacryloyloxypropylpolydimethylsiloxane in cyclopentadimethylsiloxane and the tensioning agent is a copolymer comprising a backbone including isobutyl (meth)acrylate or tert-butyl (meth)acrylate units grafted by polydimethylsiloxane grafts.

The dispersion of grafted polymer particles can be prepared by a process comprising a stage of radical copolymerization, in a liquid fatty phase corresponding to the definition given above, of one or more acrylic monomers (and optionally of one or more additional non-acrylic vinyl monomers) as defined above with one or more macromonomers as defined above.

Conventionally, the copolymerization can be carried out in the presence of a polymerization initiator. The polymerization initiators can be radical initiators. Generally, such a polymerization initiator can be chosen from organic peroxide compounds, such as dilauroyl peroxide, dibenzoyl peroxide or tert-butyl peroxy(2-ethylhexanoate), or diazo compounds, such as azobisisobutyronitrile or azobisdimethylvaleronitrile.

The reaction can also be initiated using photoinitiators or by radiation, such as UV radiation, neutrons or by plasma.

Generally, in order to carry out this process, at least a portion of the liquid fatty phase, a portion of the acrylic and/or additional vinyl monomers which will constitute, after polymerization, the insoluble backbone, all the macromonomer (which will constitute the side chains of the polymer) and a portion of the polymerization initiator are introduced into a reactor with a size appropriate to the amount of polymer which will be produced. At this stage of introduction, the reaction medium forms a relatively homogeneous medium.

The reaction medium is subsequently stirred and heated to a temperature in order to obtain polymerization of the monomers and macromonomers. After a certain time, the initially homogeneous and clear medium results in a dispersion of milky appearance. A mixture composed of the remaining portion of monomers and of the polymerization initiator is subsequently added. After an appropriate time, during which the mixture is heated with stirring, the medium stabilizes in the form of a milky dispersion, the dispersion comprising particles of stabilized polymers in the liquid fatty phase in which the particles were created, the said stabilization being due to the presence, in the polymer, of side chains soluble in the said medium.

The polymer dispersion can be present in a proportion of 3 to 95% by weight of active material in the composition, in particular from 4 to 90% by weight, indeed even from 20 to 70% by weight, with respect to the total weight of the composition.

Finally, it should be noted that the grafted polymers constituting the dispersion, improving the mechanical properties of the tensioning film, advantageously exhibit a glass transition temperature of less than or equal to 40° C.

According to the invention, the polymer dispersion described above is used in a cosmetic composition.

The composition according to the invention comprises, as was mentioned above, a fatty phase in which the polymer described above forms a dispersion.

The said fatty phase represents, for example, from 0.5 to 80% of the total weight of the composition, preferably from 1 to 55% and better still from 1 to 25%.

The composition also advantageously comprises an aqueous phase in which the tensioning agent is generally found, although it can, in an alternative form, be found in the fatty phase, depending on its nature.

Thus, the composition of the present invention can be an emulsion, in particular an oil-in-water (O/W) or water-in-oil (W/O) or multiple (W/O/W or polyol/O/W or O/W/O) emulsion, in the cream, gel, milk, paste, foam or two-phase or multiphase lotion form.

It is understood that the said cosmetic composition will comprise, in addition to the abovementioned polymer(s), a physiologically acceptable medium suitable for topical application to the skin of the face.

The said physiologically acceptable medium is generally cosmetically acceptable, that is to say that it exhibits a pleasant smell, a pleasant colour and a pleasant feel compatible with cosmetic use and does not cause discomfort (smarting, tugging, redness) capable of dissuading the user from employing it.

The composition according to the invention can also comprise ingredients commonly used in cosmetics, such as thickening agents, sequestering agents, fragrances, basifying or acidifying agents, preservatives, sunscreens, surfactants, fillers, pigments and dyes, and their mixtures.

It can also comprise anti-ageing active principles with a complementary effect to the polymers defined above, such as at least one compound chosen from desquamating agents, moisturizing agents, agents which stimulate the proliferation and/or the differentiation of keratinocytes, agents which stimulate the synthesis of collagen and/or of elastin or which prevent their decomposition, depigmenting agents, antiglycation agents, agents which stimulate the synthesis of glycosaminoglycans, dermo-decontracting agents or muscle relaxants, antioxidants and agents for combating free radicals, and their mixtures.

Of course, a person skilled in the art will take care to choose this or these optional additional compounds and/or their amount so that the advantageous properties of the corresponding composition according to the invention are not, or not substantially, detrimentally affected by the envisaged addition.

The composition is generally applied according to the usual techniques, for example by application of creams, gels, serums or lotions to the skin intended to be treated, in particular the skin of the outline of the eye. In the context of this process, the composition can, for example, be a care composition or a make-up composition, in particular a foundation.

The present invention relates, according to a second subject-matter, to a cosmetic composition comprising, in a physiologically acceptable medium suitable for topical application to the skin of the face:
from 0.1 to 20% by weight of at least one tensioning agent, with respect to the total weight of the composition, the said tensioning agent being in the form of colloidal particles of inorganic fillers; and
at least one dispersion in a liquid fatty phase of solid particles of a grafted ethylenic polymer.

The present invention relates, according to a third subject-matter, to the use of a dispersion of solid particles of an ethylenic polymer as defined above for improving the persistence of the tensioning effect provided by a tensioning agent, the said tensioning agent being as defined above.

The persistence of the tensioning effect is quantified by tests appearing in the experimental part of this description.

According to a fourth subject-matter, the present invention relates to the use of a dispersion of solid particles of an ethylenic polymer in a liquid fatty phase, such as a volatile oil as defined above, the said polymer being as defined above, in a cosmetic composition comprising, as tensioning agent, an aqueous dispersion of colloidal inorganic particles, in particular of silica, for preventing whitening of the skin while providing an improvement in the persistence of the tensioning effect induced by the tensioning agent.

The invention will now be described with reference to the following examples, given by way of illustration and without limitation.

### BRIEF DESCRIPTION OF THE FIGURES

The single FIGURE illustrates a curve representing the force F (in newtons, N) as a function of the displacement d (in mm), the said curve being used to quantify the persistence of the tensioning effect of the compositions of the invention.

### DETAILED ACCOUNT OF SPECIFIC EMBODIMENTS

Various polymers and formulations were prepared incorporating polymers as defined above in combination with tensioning agents and were tested in order to demonstrate the improvement in the persistence of the tensioning effect induced by the use of such polymers in these compositions.

Before giving the detailed account of the preparations of polymers and of formulations, two protocols will be described which make it possible to quantify the persistence of the tensioning effect induced by the abovementioned polymer/tensioning agent combination.

*Protocols for Quantifying the Persistence of the Tensioning Effect

*First Protocol.

Principle of the Test

The persistence properties which are being sought for are achieved by virtue of the introduction of compounds acting as strengthening agents, these compounds being the grafted ethylenic polymers in the dispersion form as defined above. The strengthening potential of the compounds which are used was quantified from the measurement of the breaking strength of the materials (in the present case, an anti-wrinkle cream).

The test consists in stressing under compression, to breaking point, the material deposited at the surface of a flexible and deformable foam. The use of this support made of foam makes it possible to impose a significant deformation on the material deposited at the surface and thus to quantify its breaking strength. The compressive mechanical stress is exerted using a cylindrical probe with a diameter of 1 mm, the rate of displacement of the probe being 0.1 mm/s. The test is carried out using a TA.XT2i texture analyser sold by Stable Micro System. A curve of force F (in N) as a function of the displacement d (in mm) is thus obtained, from which it is possible to determine the breaking point of the material F_{break} (N) (in accordance with what is represented in the single figure).

Two parameters are selected for quantifying the breaking strength of the material:

\textcircled{1} F_{break} (N): force at break

\textcircled{2} W_{break} (J/m²): energy at break: area under the curve Force=f(d)/area of the probe

The substrate is composed of a neoprene foam with a thickness of 13 mm. The material (anti-wrinkle composition of the invention) is deposited on this substrate so as to obtain, after drying for 24 h, a film with a thickness of 15 to 30 μm. The deposited layers were produced using a film drawer which deposits 650 μm wet (that is to say, before drying).

*Second Protocol

This second protocol is targeted at quantifying, by an in vitro retractation test, the persistent tensioning effect induced by the compositions of the invention.

More specifically, this protocol consists in quantifying in vitro the persistent tensioning effect of a composition deposited on a substrate made of elastomer having a modulus of the order of 20 MPa and with a thickness of 100 μim.

In a first step, a composition comprising a tensioning agent at 20% by weight is deposited in an amount of 30 μl on the substrate in the form of a rectangular test specimen (10×40 mm) made of elastomer. After drying at 22±3° C. and at 40±10% relative humidity for 3 h, the tension exerted by this deposited layer on the substrate and consequently the tensioning effect is directly related to the reduction in the width at the centre of the test specimen. This tensioning effect (TE) is then quantified in the following way:
TE=(L₀−L₃ₕ/L₀)×100 (expressed as %)
with:
L₀ representing the initial width of the test specimen, that is to say 10 mm;
L₃ₕ representing the width of the test specimen after drying for 3 hours.

In a second step, the test specimen is subsequently drawn manually by 50% (the length of the test specimen changing from 40 mm to 60 mm) and, after returning to its initial length, its width is again measured so as to quantify the persistence of the tensioning effect after stressing (TE_{P}).

The quantity TE_{P} is quantified in the following way:
TEₚ=(L₀−L_{100%}/L₀)×100 (expressed as %)
with:
L₀ representing the initial width of the test specimen, that is to say 10 mm;
L_{100%} representing the width of the test specimen after the latter has been subjected to the deformation defined above and returning to the initial length.

The greater the quantity TEₚ, the greater the persistence of the tensioning effect.

### COMPARATIVE EXAMPLE

This example illustrates a cosmetic composition comprising a tensioning agent in the form of an aqueous colloidal silica dispersion (Cosmo S40), the said composition being devoid of grafted ethylenic polymer in the dispersion form in accordance with the present invention.

The composition is as follows:

| Constituents | Amount | |
|---|---|---|
| Glyceryl stearate and PEG-100 stearate | 2 | g |
| Dimyristyl tartrate and cetearyl alcohol and | 1.50 | g |
| C₁₂-C₁₅-pareth-7 and PPG-25-laureth-25 | | |
| Cyclohexasiloxane | 10 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from Clariant) | 0.40 | g |
| Xanthan gum | 0.20 | g |
| Cosmo S40 (aqueous colloidal silica dispersion) | 17.10 | g |

The composition is prepared in the following way:

The phase composed of the water, the phenoxyethanol, the sequestering agent and the xanthan gum is heated to 75° C. The thickening polymer (that is to say, the polyacrylamide) is subsequently incorporated therein. The mixture is stirred until a homogeneous gel is obtained.

The phase composed of the glyceryl stearate, the PEG-100 stearate, the dimyristyl tartrate, the cetearyl alcohol, the C₁₂-C₁₅-pareth-7, the PPG-25-laureth-25, the cyclohexasiloxane and the stearyl alcohol is heated to 75° C. This phase is subsequently incorporated in the preceding phase to produce an emulsion. The aqueous colloidal silica dispersion is subsequently incorporated in the emulsion at 40-45° C. and stirring is maintained until completely cooled.

### EXAMPLE 1

This example illustrates the preparation and the use of a polymer forming a dispersion of particles in a carbon solvent, the said polymer being obtained by polymerization of methyl acrylate and of the macromonomer polyethylene/polybutylene methacrylate (Kraton L-1253) in isododecane.

*Preparation of the Dispersion

50 g of heptane, 50 g of isododecane, 7 g of methyl acrylate, 3 g of macromonomer of the poly-ethylene/polybutylene methacrylate type (Kraton L-1253) and 0.8 g of tert-butyl peroxy(2-ethylhexanoate) (Trigonox 21S) are charged to a 500 ml reactor.

The reaction mixture is stirred and heated from ambient temperature to 90° C. over 1 hour. After 15 minutes at 90° C., a change in appearance of the reaction medium is observed, the latter changing from a transparent appearance to a milky appearance. Heating is maintained with stirring for an additional 15 minutes and then a mixture composed of 40 g of methyl acrylate and 0.5 g of Trigonox 21S is added dropwise over 1 hour.

The mixture is subsequently left heating at 90° C. for 4 hours and then the heptane is distilled from the reaction medium.

On conclusion of this distillation operation, a dispersion thus prepared of polymer particles is obtained which is stable in isododecane. The characteristics of this polymer and of the particles formed by the said polymer are as follows:
Particle sizing: 46 mm, carried out with a Malvern Autosizer Lo-C at 25° C.;
Solids content: 50% in isododecane, carried out with a thermobalance;
Weight-average molecular weight Mw: 119 200;
Number-average molecular weight Mn: 31 900;
Polydispersity index (Mw/Mn): 3.74;
Molecular weight of the macromonomer used Mw: 4000;
Glass transition temperature <40° C. (measured by dynamic mechanical thermal analysis (DMTA)).
   *Preparation of the Composition

The composition comprises the following ingredients:

| Constituents | Amount | |
|---|---|---|
| Glyceryl stearate and PEG-100 stearate | 2 | g |
| Dimyristyl tartrate and cetearyl alcohol and | 1.50 | g |
| C₁₂-C₁₅-pareth-7 and PPG-25-laureth-25 | | |
| Cyclohexasiloxane | 5.39 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from Clariant) | 0.40 | g |
| Xanthan gum | 0.20 | g |
| Cosmo S40 (aqueous colloidal silica dispersion) | 17.10 | g |
| Polymer dispersion prepared above | 4.61 | g |

The composition of this example is prepared in the same way as that of the above comparative example, this preparation additionally comprising the incorporation of the polymer prepared above at 40-45° C. in the emulsion after the introduction of the aqueous colloidal silica dispersion.

### EXAMPLE 2

This example illustrates the preparation and the use of a polymer forming a dispersion of particles in a silicone solvent, the said polymer being obtained by polymerization of methyl acrylate and of the macromonomer corresponding to monomethacryloyloxypropylpolydimethylsiloxane in cyclopentadimethylsiloxane.

*Preparation of the Dispersion

50 g of heptane, 50 g of cyclopentadimethylsiloxane, 7.5 g of methyl acrylate, 2.5 g of macromonomer of the monomethacryloyloxypropylpolydimethylsiloxane type and 0.8 g of tert-butyl peroxy(2-ethylhexanoate) (Trigonox 21S) are charged to a 500 ml reactor.

The reaction mixture is stirred and heated from ambient temperature to 90° C. over 1 hour. After 15 minutes at 90° C., a change in appearance of the reaction medium is observed, the latter changing from a transparent appearance to a milky appearance. Heating is maintained with stirring for an additional 15 minutes and then a mixture composed of 40 g of methyl acrylate and 0.5 g of Trigonox 21S is added dropwise over 1 hour.

The mixture is subsequently left heating at 90° C. for 4 hours and then the heptane is distilled from the reaction medium.

On conclusion of this distillation operation, a dispersion thus prepared of polymer particles is obtained which is stable in cyclopentadimethylsiloxane. The characteristics of this polymer and of the particles formed by the said polymer are as follows:
Particle sizing: 162 mm, carried out with a Malvern Autosizer Lo-C at 25° C.;
Solids content: 51.4% in cyclopentadimethylsiloxane, carried out with a thermobalance;
Weight-average molecular weight Mw: 104 400;
Number-average molecular weight Mn: 28 500;
Polydispersity index (Mw/Mn): 3.67;
Molecular weight of the macromonomer used Mw: 5000;
Glass transition temperature <40° C. (measured by dynamic mechanical thermal analysis (DMTA)).
   *Preparation of the Composition

The composition comprises the following ingredients:

| Constituents | Amount | |
|---|---|---|
| Glyceryl stearate and PEG-100 stearate | 2 | g |
| Dimyristyl tartrate and cetearyl alcohol and | 1.50 | g |
| C₁₂-C₁₅-pareth-7 and PPG-25-laureth-25 | | |
| Cyclohexasiloxane | 5.13 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from Clariant) | 0.40 | g |
| Xanthan gum | 0.20 | g |
| Cosmo S40 (aqueous colloidal silica dispersion) | 17.10 | g |
| Polymer dispersion prepared above | 4.87 | g |

The composition of this example is prepared in the same way as that of the above comparative example, this preparation additionally comprising the incorporation of the polymer prepared above at 40-45° C. in the emulsion after the introduction of the aqueous colloidal silica dispersion.

### EXAMPLE 3

This example illustrates the preparation of a dispersion of solid particles of a polymer in a carbon solvent which is obtained by polymerization of methyl acrylate, of acrylic acid and of the macromonomer polyethylene/polybutylene methacrylate (Kraton L-1253). This dispersion is used as tensioning agent in the context of this invention.

The procedure for the preparation of this specific dispersion is as follows:

50 g of heptane, 50 g of isododecane, 3.5 g of methyl acrylate, 2.5 g of acrylic acid, 4 g of the macromonomer polyethylene/polybutylene methacrylate (Kraton L-1253) and 0.8 g of tert-butyl peroxy(2-ethylhexanoate) (Trigonox 21S) are charged to a 500 ml reactor.

The reaction mixture is stirred and heated from ambient temperature to 90° C. over 1 hour. After 15 minutes at 90° C., a change in appearance of the reaction medium is observed, the latter changing from a transparent appearance to a milky appearance. Heating is maintained with stirring for an additional 15 minutes and then a mixture composed of 17.5 g of methyl acrylate, 22.5 g of acrylic acid and 0.5 g of Trigonox 21S is added dropwise over 1 hour.

The mixture is subsequently left heating at 90° C. for 4 hours and then the heptane is distilled from the reaction medium.

On conclusion of this distillation operation, a dispersion thus prepared of polymer particles is obtained which is stable in isododecane. The characteristics of this polymer and of the particles formed by the said polymer are as follows:
Particle sizing: 63 mm, carried out with a Malvern Autosizer Lo-C at 25° C.;
Solids content: 53.6% in isododecane, carried out with a thermobalance;
Molecular weight of the macromonomer used Mw: 4000.

### EXAMPLE 4

Demonstration of the Persistent Tensioning Effect According to the First Protocol

The protocol for quantifying the persistence of the tensioning effect as defined above was carried out for the three compositions of the Comparative Example and of Examples 1 and 2.

This protocol is targeted at quantifying the strengthening potential of the polymers in the form of dispersions of Example 1 (in isododecane) and of Example 2 (in cyclopentadimethylsiloxane) once introduced into an anti-wrinkle composition.

The results obtained are combined in the table below.

| | | W_{break} |
|---|---|---|
| | F_{break} (N) | (J/m²) |
| Comparative Example | 0.18 ± 0.02 | 19 ± 3 |
| Example 1 (silica CS40 7% + 2.5% | 0.40 ± 0.01 | 146 ± 11 |
| of the dispersion) | | |
| Example 2 (silica CS40 7% + 2.5% | 0.41 ± 0.01 | 131 ± 5 |
| of the dispersion) | | |

These results demonstrate the strengthening role of the two dispersions studied in the presence of a tensioning agent. This strengthening role is illustrated by an increase in the force and in the energy at break.

### EXAMPLE 5

Demonstration of the Persistent Tensioning Effect According to the Second Protocol

The second protocol was carried out for the following compositions:
a composition composed solely of a dispersion of a polymer of Example 3 in isododecane (entitled Composition a);
a composition composed of a mixture in isododecane in a 90/10 ratio of a dispersion of Example 3 and of a dispersion of Example 1 (entitled Composition b);
a composition composed solely of a tensioning agent in dispersion in cyclopentadimethylsiloxane, this tensioning agent being a copolymer of isobutyl methacrylate, of acrylic acid and of a CTFA silicone macromonomer (as defined above) at 23% by weight (sold under the reference SA 70 by 3M) (entitled Composition c);
a composition composed of a mixture in cyclopentadimethylsiloxane in a 90/10 ratio of a tensioning agent SA70 and of a dispersion as prepared in Example 2 (Composition d).

The results obtained according to this second protocol are combined in the following table.

| | TE(%) | TE_{P}(%) |
|---|---|---|
| Composition a | 42 ± 5 | 0 |
| Composition b | 62 ± 5 | 45 ± 6 |
| Composition c | 80 ± 5 | 20 ± 4 |
| Composition d | 80 ± 5 | 50 ± 5 |

It can be seen that the compositions comprising a dispersion in accordance with the present invention exhibit a greater quantity TE_{P} than the compositions not comprising such a dispersion, which means that the dispersions of the invention bring about a persistence in the tensioning effect produced by the tensioning agent.

### EXAMPLE 6

Effect on the Whitening of the Skin

The cosmetic compositions corresponding to the Comparative Example and Example 2 above were spread using a mechanical film drawer over a contrast card (Prufkarte type 24/5-250 cm²) sold by Erichsen (thickness of the film: 30 μm). The compositions were subsequently dried at a temperature of 20° C. for 3 hours and photographs of the treated areas were taken.

In the case of the composition of the Comparative Example, the appearance of unsightly white deposits on the treated area was noticed. In the case of the composition of Example 2 according to the invention, such unsightly deposits are absent.

### REFERENCES CITED

[1] FR-A-2 758 083;
[2] U.S. Pat. No. 6,139,322;
[3] U.S. Pat. No. 6,465,001;
[4] U.S. Pat. No. 5,349,003;
[5] EP-1 038 519;
[6] FR-2 819 429;
[7] FR-03/11346;
[8] Food Gels, Peter Harris, Elsevier 1989, Chap. 3;
[9] Food Gels, Peter Harris, Elsevier 1989, Chap. 1;
[10] Food Gels, Peter Harris, Elsevier 1989, Chap. 6;
[11] FR-2 829 025;
[12] Kirk-Othmer Encyclopedia Of Chemical Technology, 3rd edition, volume 21, pp.492-507, Wiley Interscience, 1983;
[13] Light Scattering by Small Particles, Wiley, New York, 1957, Chap. 9 and 10;
[14] Polymer Handbook, 3rd edition, Chapter VII, pp.519-559;
[15] J. Paint Technol., 39, 105 (1967);
[16] EP 895 467;
[17] EP 96 459;
[18] Polymer Letters, Vol. 5, pages 477-481 (1967);
[19] U.S. Pat. No. 5,625,005.

1-59. (canceled)

60. A cosmetic process for softening the wrinkles of wrinkled skin comprising applying to said wrinkled skin a cosmetic composition comprising, in a physiologically acceptable medium suitable for topical application to the skin of the face:
from 0.1 to 20% by weight, based on the total weight of the composition, of at least one tensioning agent, and
at least one dispersion of solid particles of a grafted ethylenic polymer in a liquid fatty phase.

61. The cosmetic process according to claim 60, in which said tensioning agent is present at a content ranging from 1 to 10% of the total weight of the composition.

62. The cosmetic process according to claim 60, in which said dispersion is present in the composition at a content ranging from 0.01 to 20%.

63. The cosmetic process according to claim 60, in which said liquid fatty phase is present in the composition at a content ranging from 0.5 to 80% of the total weight of the composition.

64. The cosmetic process according to claim 60, in which said tensioning agent is an agent producing, at a concentration of 7% in water, a retraction of the isolated stratum corneum, measured with an extensometer, of more than 1% at 30° C. under a relative humidity of 40%.

65. The cosmetic process according to claim 60, in which said tensioning agent is chosen from the group consisting of synthetic polymers, polymers of natural origin, mixed silicates, wax microparticles, colloidal particles of inorganic fillers, and mixtures thereof.

66. The cosmetic process according to claim 65, in which said synthetic polymers are chosen from:
polyurethane polymers and copolymers;
acrylic polymers and copolymers;
polymers of sulphoisophthalic acid;
grafted silicone polymers;
water-soluble or water-dispersible polymers comprising water-soluble or water-dispersible units and LCST units;
non-elastomeric and water-insoluble film-forming linear ethylenic block polymers exhibiting a dynamic storage modulus E′ at 1 Hz and 22° C. of greater than 200 MPa;
grafted ethylenic polymers as a dispersion of solid particles in a liquid fatty phase exhibiting a glass transition temperature of greater than 40° C.; and
mixtures thereof.

67. The cosmetic process according to claim 65, in which said polymers of natural origin are chosen from plant proteins and plant protein hydrolysates; polysaccharides of plant origin in the form of microgels; latexes of plant origin; and mixtures thereof.

68. The cosmetic process according to claim 60, in which said grafted ethylenic polymer comprises a backbone which is insoluble in said liquid fatty phase and a part which is soluble in said liquid fatty phase composed of side chains covalently bonded to said backbone.

69. The cosmetic process according to claim 60, in which said grafted ethylenic polymer is a grafted acrylic polymer.

70. The cosmetic process according to claim 69, in which said grafted acrylic polymer is obtained by radical polymerization in said liquid fatty phase:
of at least one acrylic monomer and optionally at least one additional non-acrylic vinyl monomer as an insoluble backbone; and
of at least one macromonomer comprising a polymerizable end group, said macromonomer forming side chains on said backbone, said macromonomer having a weight-average molecular weight of greater than or equal to 200 and the content of polymerized macromonomer representing from 0.05 to 20% by weight of said grafted acrylic polymer.

71. The cosmetic process according to claim 70, in which said acrylic monomer or monomers are chosen from monomers, the homopolymer of which is insoluble in said liquid fatty phase and is in the solid form in said liquid fatty phase at a concentration of greater than or equal to 5% by weight at an ambient temperature of 20° C.

72. The cosmetic process according to claim 70, in which said acrylic monomer or monomers are chosen from the group consisting of:
(meth)acrylates of formula (IV): 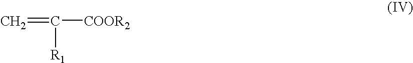
in which:
R₁ denotes a hydrogen atom or a methyl group; and
R₂ represents a group chosen from:
a linear or branched alkyl group comprising from 1 to 6 carbon atoms, optionally comprising in its chain one or more heteroatoms chosen from O, N and S; and/or optionally comprising one or more substituents chosen from —OH, halogen atoms selected from the group consisting of F, Cl, Br, and I, and —NR′R″ wherein R′ and R″, which are identical or different, are chosen from linear or branched alkyl groups comprising from 1 to 4 carbon atoms; and/or optionally being substituted by at least one polyoxyalkylene group, said polyoxyalkylene group being composed of the repetition of 5 to 30 oxyalkylene units; and
a cyclic alkyl group comprising from 3 to 6 carbon atoms, optionally comprising in its chain one or more heteroatoms chosen from O, N and S and optionally comprising one or more substituents chosen from the group consisting of —OH and halogen atoms;
(meth)acrylamides of formula (V): 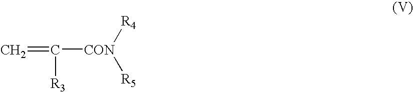
in which:
R₃ denotes a hydrogen atom or a methyl group; and
R4 and R₅, which are identical or different, represent a hydrogen atom or a linear or branched alkyl group comprising from 1 to 6 carbon atoms optionally comprising one or more substituents chosen from —OH, halogen atoms and —NR′R″ wherein R′ and R″, which are identical or different, are chosen from linear or branched alkyl groups comprising from 1 to 4 carbon atoms; or
R₄ represents a hydrogen atom and R₅ represents a 1,1 dimethyl-3-oxobutyl group; and
(meth)acrylic monomers comprising at least one carboxylic, phosphoric or sulphonic acid functional group;
said monomers optionally in the form of salts.

73. The cosmetic process according to claim 70, in which said additional non-acrylic vinyl monomer or monomers are chosen from the group consisting of:
vinyl esters of formula (VI):
R₆—COO—CH═CH₂ (VI)
in which:
R₆ represents a linear or branched alkyl group comprising from 1 to 6 carbon atoms or a cyclic alkyl group comprising from 3 to 6 carbon atoms and/or an aromatic group;
non-acrylic vinyl monomers comprising at least one carboxylic or sulphonic acid functional group and the salts thereof;
non-acrylic vinyl monomers comprising at least one tertiary amine functional group; and
mixtures thereof.

74. The cosmetic process according to claim 70, in which said acrylic monomer or monomers represent from 50 to 100% by weight of the mixture composed of the acrylic monomer or monomers and said optional non-acrylic vinyl monomer or monomers.

75. The cosmetic process according to claim 70, in which said grafted acrylic polymer does not comprise additional non-acrylic vinyl monomers.

76. The cosmetic process according to claim 70, in which said macromonomer comprises, at one of its ends, a polymerizable end group chosen from a vinyl group and a (meth)acrylate group.

77. The cosmetic process according to claim 70, in which said macromonomer exhibits a weight-average molecular weight (Mw) ranging from 200 to 100 000.

78. The cosmetic process according to claim 70, in which the polymerized macromonomer represents from 0.1 to 15% by weight of the total weight of said polymer.

79. The cosmetic process according to claim 70, in which said macromonomer is chosen from macromonomers, the homopolymer of which is completely dissolved in said liquid fatty phase at a concentration of greater than or equal to 5% by weight and at an ambient temperature of 20° C.

80. The cosmetic process according to claim 60, in which the said liquid fatty phase comprises at least one non-aqueous liquid compound chosen from the group consisting of:
liquid organic compounds having an overall solubility parameter according to the Hansen solubility space of less than or equal to 18 (MPa)^{1/2};
monoalcohols having an overall solubility parameter according to the Hansen solubility space of less than or equal to 20 (MPa)^{1/2}; and
mixtures thereof.

81. The cosmetic process according to claim 80, in which said liquid fatty phase is a non-silicone fatty phase.

82. The cosmetic process according to claim 81, in which said non-silicone fatty phase comprises at least 50% by weight of at least one non-silicone liquid organic compound chosen from:
non-silicone liquid organic compounds having an overall solubility parameter according to the Hansen solubility space of less than or equal to 18 (MPa)^{1/2};
liquid monoalcohols having an overall solubility parameter according to the Hansen solubility space of less than or equal to 20 (MPa)^{1/2}; and
mixtures thereof.

83. The cosmetic process according to claim 82, in which said non-silicone liquid compound having an overall solubility parameter according to the Hansen solubility space of less than or equal to 18 (MPa)^{1/2}, is chosen from:
optionally branched, carbon, hydrocarbon or fluorinated, natural or synthetic oils, alone or as a mixture;
optionally volatile, linear, branched and/or cyclic alkanes;
linear, branched or cyclic esters having at least 6 carbon atoms;
ethers having at least 6 carbon atoms; and
ketones having at least 6 carbon atoms.

84. The cosmetic process according to claim 82, in which said monoalcohols having an overall solubility parameter according to the Hansen solubility space of less than or equal to 20 (MPa)^{1/2} are chosen from the group consisting of saturated and unsaturated liquid aliphatic fatty monoalcohols having at least 6 carbon atoms.

85. The cosmetic process according to claim 82, in which said non-silicone liquid fatty phase comprises less than 50% by weight of silicone liquid organic compounds.

86. The cosmetic process according to claim 82, in which said non-silicone liquid fatty phase does not comprise silicone liquid organic compounds.

87. The cosmetic process according to claim 82, in which the macromonomer or macromonomers are carbon macromonomers.

88. The cosmetic process according to claim 87, in which said carbon macromonomer is chosen from:
(i) linear or branched C₈-C₂₂ alkyl (meth)acrylate homopolymers and copolymers exhibiting a polymerizable end group chosen from vinyl and (meth)acrylate groups; and
(ii) polyolefins having an end group comprising ethylenic unsaturation.

89. The cosmetic process according to claim 87, in which said carbon macromonomer is chosen from the group consisting of:
(i) poly(2-ethylhexyl acrylate) macromonomers having a mono(meth)acrylate end; poly(dodecyl acrylate) macromonomers having a mono(meth)acrylate end; poly(dodecyl methacrylate) macromonomers having a mono(meth)acrylate end; poly(stearyl acrylate) macromonomers having a mono(meth)acrylate end; and poly(stearyl methacrylate) macromonomers having a mono(meth)acrylate end; and
(ii) polyethylene macromonomers, polypropylene macromonomers, polyethylene/polypropylene copolymer macromonomers, polyethylene/polybutylene copolymer macromonomers, polyisobutylene macromonomers, polybutadiene macromonomers, polyisoprene macromonomers, polybutadiene macromonomers, and poly(ethylene/butylene)-polyisoprene macromonomers, wherein all of said macromonomers have a (meth)acrylate end group.

90. The cosmetic process according to claim 88, in which said carbon macromonomer is chosen from the group consisting of:
(i) poly(2-ethylhexyl acrylate) macromonomers having a mono(meth)acrylate end, and poly(dodecyl acrylate) macromonomers having a mono(meth)acrylate end; and
(ii) poly(ethylene/butylene) methacrylate.

91. The cosmetic process according to claim 60, in which said dispersion is a dispersion obtained by the polymerization of methyl acrylate and the macromonomer polyethylene/polybutylene methacrylate in isododecane and said tensioning agent is a colloidal silica dispersion.

92. The cosmetic process according to claim 60, in which said dispersion is a dispersion obtained by the polymerization of methyl acrylate and the macromonomer polyethylene/polybutylene methacrylate in isododecane and said tensioning agent is a dispersion obtained by the polymerization in isododecane of methyl acrylate, acrylic acid and the macromonomer polyethylene/polybutylene methacrylate.

93. The cosmetic process according to claim 80, in which said liquid fatty phase is a silicone liquid fatty phase.

94. The cosmetic process according to claim 93, in which said silicone liquid fatty phase comprises at least 50% by weight of at least one silicone liquid organic compound chosen from silicone liquid organic compounds having an overall solubility parameter according to the Hansen solubility space of less than or equal to 17 (MPa)^{1/2}.

95. The cosmetic process according to claim 93, in which said liquid fatty phase comprises a volatile silicone oil.

96. The cosmetic process according to claim 95, in which said volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, and mixtures thereof.

97. The cosmetic process according to claim 93, in which said liquid fatty phase comprises a non-volatile silicone oil.

98. The cosmetic process according to claim 97, in which said non-volatile silicone oil is chosen from polydialkylsiloxanes; polydimethylsiloxanes comprising pending alkyl, alkoxy or phenyl groups or alkyl, alkoxy or phenyl groups at the end of the silicone chain, which groups have from 2 to 24 carbon atoms; phenylated silicones;
polysiloxanes modified with fatty acids, fatty alcohols or polyoxyalkylenes; aminated polysiloxanes; polysiloxanes comprising hydroxyl groups; and fluorinated polysiloxanes comprising a pending fluorinated group or a fluorinated group at the end of the silicone chain having from 1 to 12 carbon atoms, all or part of the hydrogens of which are substituted by fluorine atoms; and mixtures thereof.

99. The cosmetic process according to claim 93, in which said liquid fatty phase comprises less than 50% by weight of non-silicone liquid organic compounds.

100. The cosmetic process according to claim 93, in which said liquid fatty phase does not comprise non-silicone liquid organic compounds.

101. The cosmetic process according to claim 93, in which the macromonomer is a silicone macromonomer.

102. The cosmetic process according to claim 101, in which said silicone macromonomer is an organopolysiloxane macromonomer.

103. The cosmetic process according to claim 93, in which said grafted acrylic polymer is obtained by radical polymerization in said liquid fatty phase:
of a main acrylic monomer which forms an insoluble backbone of said grafted acrylic polymer, said monomer chosen from C₁-C₃ alkyl (meth)acrylates, alone or as a mixture, and optionally one or more additional acrylic monomers chosen from the group consisting of acrylic acid, methacrylic acid and alkyl (meth)acrylates of formula (VII): 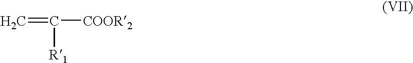
in which:
R′₁ denotes a hydrogen atom or a methyl group; and
R′₂ represents:
a linear or branched alkyl group comprising from 1 to 6 carbon atoms, said group comprising, in its chain, one or more oxygen atoms and/or comprising one or more substituents chosen from —OH, halogen atoms and —NR′R″ wherein R′ and R″, which are identical or different, are chosen from linear or branched C₁-C₃ alkyls;
a cyclic alkyl group comprising from 3 to 6 carbon atoms, said group comprising in its chain, one or more oxygen atoms and/or comprising one or more substituents chosen from —OH and halogen atoms;
and their salts, in order to form the said insoluble backbone; and a silicone macromonomer.

104. The cosmetic process according to claim 103, in which R′₂ is selected from the group consisting of methoxyethyl, ethoxyethyl, trifluoroethyl, 2-hydroxyethyl, 2-hydroxypropyl, dimethylaminoethyl, diethylaminoethyl and dimethylaminopropyl.

105. The cosmetic process according to claim 103, in which said main acrylic monomer is chosen from the group consisting of methyl (meth)acrylate, ethyl (meth)acrylate, n-propyl meth(acrylate), isopropyl (meth)acrylate, and mixtures thereof.

106. The cosmetic process according to claim 105, in which said acrylic monomer is chosen from the group consisting of methyl acrylate, methyl methacrylate and ethyl methacrylate.

107. The cosmetic process according to claim 103, in which said additional acrylic monomer is chosen from the group consisting of (meth)acrylic acid, methoxyethyl (meth)acrylate, ethoxyethyl (meth)acrylate, trifluoroethyl methacrylate, dimethylaminoethyl methacrylate, diethylaminoethyl methacrylate, 2-hydroxypropyl (meth)acrylate, 2-hydroxyethyl (meth)acrylate, their salts and mixtures thereof.

108. The cosmetic process according to claim 107, in which said additional acrylic monomer is chosen from the group consisting of acrylic acid and methacrylic acid.

109. The cosmetic process according to claim 103, in which said silicone macromonomer is chosen from polydimethylsiloxanes comprising a mono(meth)acrylate end group.

110. The cosmetic process according to claim 109, in which said silicone macromonomer corresponds to the formula (VIII): 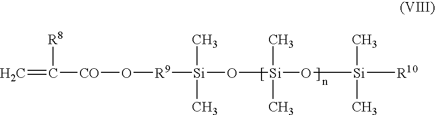
in which:
R⁸ represents a hydrogen atom or a methyl group;
R⁹ represents a divalent hydrocarbon group having from I to 10 carbon atoms and optionally comprising one or two ether —O— bonds;
R¹⁰ represents an alkyl group having from 1 to 10 carbon atoms; and
n represents an integer ranging from 1 to 300.

111. The cosmetic process according to claim 94, in which the dispersion is obtained by the polymerization of methyl acrylate and the macromonomer monomethacryloyloxypropylpolydimethylsiloxane in cyclopentadimethylsiloxane and the tensioning agent is a colloidal silica dispersion.

112. The cosmetic process according to claim 60, in which said grafted polymer has a weight-average molecular weight (Mw) of between 10,000 and 300,000.

113. The cosmetic process according to claim 60, in which said grafted polymer particles have a mean size ranging from 10 to 400 nm.

114. The cosmetic process according to claim 60, in which said composition is applied to the outline of the eye.

115. The cosmetic process according to claim 60, in which said composition is a care composition or a make-up composition.

116. The cosmetic composition comprising, in a physiologically acceptable medium suitable for topical application to the skin of the face:
from 0.1 to 20% by weight based on the total weight of the composition of at least one tensioning agent in the form of colloidal particles of inorganic fillers; and
at least one dispersion of solid particles of a grafted ethylenic polymer in a liquid fatty phase.

117. A method of using a dispersion of solid particles of a grafted ethylenic polymer as defined according to claim 60 for improving the persistence of the tensioning effect provided by said tensioning agent.

118. A method of using a dispersion of solid particles of a grafted ethylenic polymer as defined according to claim 60 in a cosmetic composition comprising, as a tensioning agent, an aqueous dispersion of colloidal inorganic particles, for preventing whitening of the skin.
